(19) United States
(12) Patent Application Publication
(10) Pub. No.: US 2025/0345660 A1
(43) Pub. Date: Nov. 13, 2025

(54) DYNAMIC HEALTH AND FITNESS MONITORING SYSTEM TO IMPROVE BODY SUPPORTING DEVICES

(71) Applicant: Intel Corporation, Santa Clara, CA (US)

(72) Inventors: Cornelius Buerkle, Karlsruhe (DE); Fabian Oboril, Karlsruhe (DE)

(21) Appl. No.: 19/278,026

(22) Filed: Jul. 23, 2025

(51) Int. Cl. A63B 24/00; A63B 21/00

(52) U.S. Cl. CPC A63B 24/0087; A63B 21/00181; A63B 24/0062; A63B 24/0075; A63B 2024/0065; A63B 2024/0093

(57) ABSTRACT

A system, including: at least one processor configured to: receive real-time data indicative of a physiological state of a user during operation of a body-support device; determine, based at least in part on the real-time data and a training plan generated from a user body model representing a current physiological state of the user, a target level of physical support for the user; generate a control signal corresponding to the target level of physical support; and dynamically adjust the control signal in response to detected changes in the user's physiological state, as indicated by the real-time data, during operation of the body-support device; and an interface configured to transmit the control signal to the body-support device to dynamically provide the target level of physical support to the user.

### TECHNICAL FIELD

Aspects described herein generally relate to systems and methods for adaptive physical support, and more particularly to real-time adjustment of body-support devices based on dynamic monitoring and analysis of a user's physiological state, fitness, and fatigue.

### BACKGROUND

Human movement is increasingly assisted by technical solutions, such as electrically-assisted bicycles (e-bikes) and exoskeletons, which are utilized in sports, industrial, healthcare, and rehabilitation settings. These body-support devices can reduce the risk of overload injuries, support individuals with physical limitations, and enable broader participation in physical activities.

Despite these benefits, reliance on assistive technologies introduces new challenges. Many individuals do not engage in enough physical activity to maintain fitness, leading to issues such as back pain, obesity, and related illnesses. Improper use of body-support devices may further reduce muscle engagement and overall fitness.

Current body-support systems are typically configured statically, based on generic profiles or predefined training plans, and do not account for real-time changes in the user's physiological state, such as fatigue or temporary injuries. While some physiological data may be collected by wearable devices, this information is not effectively integrated to dynamically adjust support levels. As a result, existing solutions may provide either insufficient or excessive support, which can lead to injury or diminish health benefits.

### BRIEF DESCRIPTION OF THE FIGURES

FIG. 1 illustrates a schematic block diagram of a system for real-time adaptive physical support based on a user's physiological state, in accordance with aspects of the disclosure.

FIG. 2 illustrates a block diagram of the health adviser agent system architecture, in accordance with aspects of the disclosure.

FIG. 3 illustrates a block diagram of the activity agent system architecture, in accordance with aspects of the disclosure.

FIG. 4 presents a graph illustrating the relationship between heart rate, desired heart rate, desired support power, and adjuster power during an activity session, in accordance with aspects of the disclosure.

FIG. 5 illustrates a computing device, in accordance with aspects of the disclosure.

### DETAILED DESCRIPTION

The present disclosure is directed to a system configured to obtain fine-grained and dynamic fitness, fatigue, and health data of a user, enabling real-time adjustments to the support provided by a body-support device, such as an electrically-assisted bicycle or exoskeleton. The system detects weaknesses or changes in the user's physiological state during activity and adjusts the level and distribution of support in real-time.

By continuously monitoring and analyzing physiological and performance data, the system enables balancing of training efforts, ensuring that the user's fitness is maintained or improved over time. This dynamic adaptation promotes better health outcomes and provides significant benefits to users by preventing both under- and over-exertion.

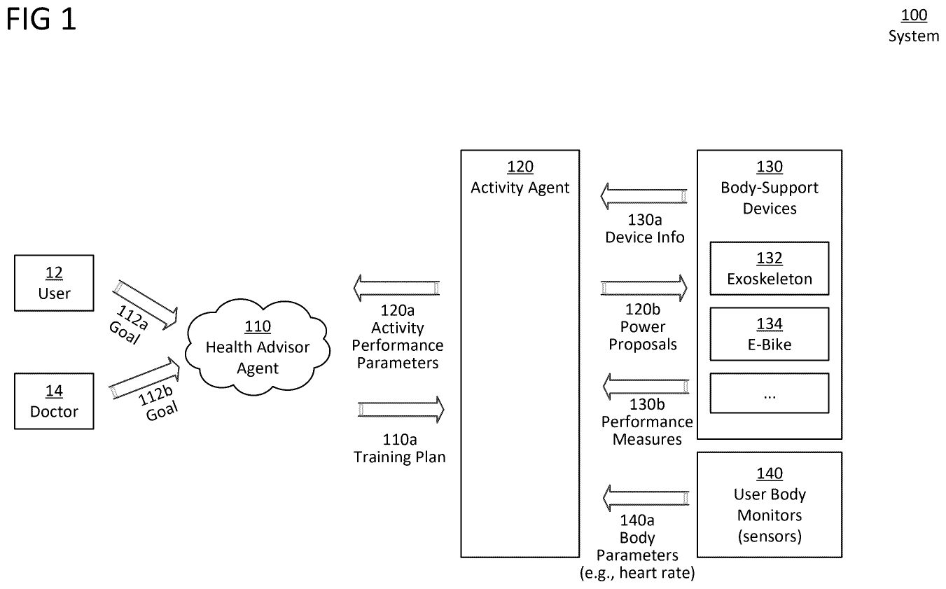

FIG. 1 illustrates a schematic block diagram of a system 100 for real-time adaptive physical support based on a user's physiological state. The system 100 includes a health adviser agent 110, an activity agent 120, and one or more body-support devices 130, such as an exoskeleton 132 or an electrically-assisted bicycle 134. The health adviser agent 110 can receive input from the user 12 and/or a doctor 14 in the form of a defined goal 112 and utilizes a user body model to generate a training plan 110a tailored to the user's current health and fitness state.

The activity agent 120 receives the training plan 110a from the health adviser agent 110 and device information 130a from at least one body-support device 130 (exoskeleton 132, e-bike 134, or the like). The activity agent 120 also receives real-time performance measures 130b, such as power and force data, from the body-support devices 130, as well as physiological data 140a from user body monitors 140 (sensors), which may include heart rate and other body parameters. Based on this information, the activity agent 120 generates power proposals 120b that specify the level and distribution of support to be provided by the body-support devices 130.

In some embodiments, user body monitors 140 may be implemented as an integrated part of the body-support device 130, or as a separate module, such as a smartwatch or other wearable sensor, communicatively coupled to the activity agent 120. The user body monitor 140 may include one or more sensors configured to measure physiological and performance parameters of the user. Such sensors may include, but are not limited to, a heart rate sensor, an electrocardiogram (ECG) sensor, a blood pressure sensor, a blood oxygenation (SpO2) sensor, a temperature sensor, a respiratory rate sensor, an electromyography (EMG) sensor, accelerometers, a gyroscope, a force sensor, and a power sensor. These sensors may be incorporated into wearable devices, such as fitness trackers or smartwatches, or embedded within the body-support device 140 itself. The data collected from these sensors is used by the activity agent 120 to assess the user's physiological state and activity performance in real time.

Performance measures 130b from the body-support devices 130 are fed back to the activity agent 120, enabling continuous monitoring and adjustment of support. The activity agent 120 also communicates activity performance parameters 120a back to the health adviser agent 110, allowing the user body model and training plan to be updated based on ongoing activity and physiological data. This closed-loop architecture enables the system 100 to dynamically and optimally adjust physical support in real time, tailored to the user's physiological state and training objectives.

As used herein, activity performance parameters 120a refers to a set of data that may include both performance measures 130b (metrics provided by the body-support device 130, such as power output or torque) and body parameters 140a (physiological metrics of the user, such as heart rate or EMG), collectively representing the user's performance and physiological state during activity.

In some embodiments, the health adviser agent 110 may be hosted remotely, such as in a cloud server or other network infrastructure. In contrast, the activity agent 120 is typically implemented locally, for example, on a user's smartphone, wearable device, or embedded controller associated with the body-support device 130. This arrangement allows the activity agent 120 to facilitate direct, low-latency interaction with hardware and sensors during physical activity. Communication between the health adviser agent 110, the activity agent 120, and the body-support devices 130 may occur via wireless protocols, such as Bluetooth, Wi-Fi, or other suitable communication technologies, enabling seamless data exchange and coordination between distributed system components.

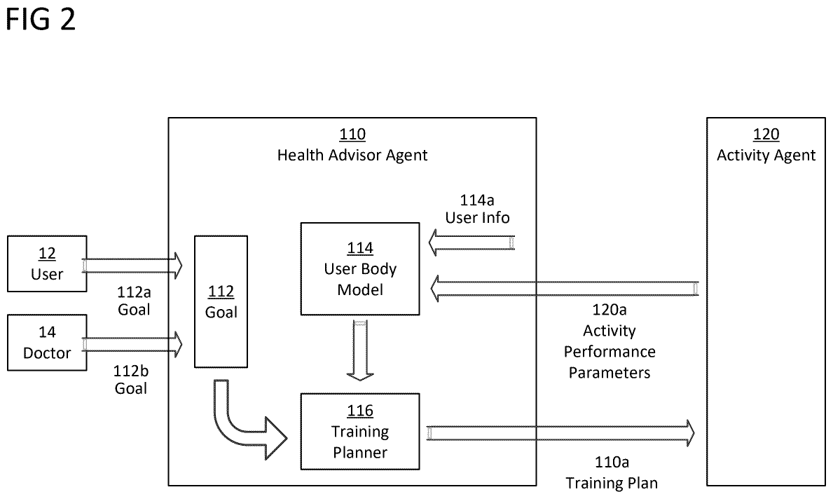

FIG. 2 illustrates a block diagram of the health adviser agent 110 system architecture. The diagram illustrates the interaction between user 12 and/or a doctor 14, and the health adviser agent 110. The user 12 and/or doctor 14 provide one or more defined goals 112 (112a and 112b, respectively), such as fitness objectives, rehabilitation targets, or health maintenance preferences, which are input to the health adviser agent 110.

The health adviser agent 110 specializes in classifying the user's health state and providing advice or a plan for the target load on the user's muscular and cardiovascular systems. As input, the health adviser agent 110 receives vital and physical information 114a of the user, such as age, weight, gender, health status, and other relevant physiological data, to estimate the current state of fitness, fatigue, and overall health. Internally, the health adviser agent 110 utilizes a user body model 114, which may be implemented as an artificial intelligence (AI) or knowledge-based model. This model can represent both generic human body mechanics and user-specific adaptations and is regularly updated using data from the user's activities and performance history. Based on this information, the user body model 114 estimates and tracks the current state of the user's cardiovascular and muscular systems, as well as fatigue and health level.

Certain user information 114a, such as weight, may be updated continuously as new data is received, while other parameters, such as height, are generally static and updated less frequently. The user body model 114 is updated accordingly to reflect both static and dynamic user characteristics.

The health adviser agent 110 may be hosted remotely, for example, in a cloud environment, and may utilize a user body model 114 implemented as a neural network-based AI model or a knowledge-based model. The user body model 114 may be user-specific, population-based, or adaptively updated. The user body model 114 may be generic in structure, but parameterized and continuously adapted to the individual user.

The user 12 and/or doctor 14 can define goals 112 to be achieved, such as increasing strength, maintaining a healthy state with reduced effort, or addressing specific rehabilitation needs. These defined goals, together with the current state of the user body, are provided to a training planner 116. The training planner 116, which can be implemented as an AI agent or reasoning model, is optimized for creating a training or stimulation plan 110a tailored to the user's body. The training plan 110a specifies target load and stimulation parameters for individual muscles, muscular systems, or the cardiovascular system, tailored to the user's current state and objectives.

The AI model may be trained using historical physiological and performance data, with the objective of predicting target support levels for various user states. Training may involve minimizing a loss function that penalizes deviations from desired physiological targets (e.g., heart rate, fatigue level) during simulated or real activities. The model may be periodically retrained or updated as new user data becomes available.

The generated training plan 110a is communicated to the activity agent 120, which is responsible for implementing the plan during physical activity. Additionally, the activity agent 120 provides activity performance parameters 120a back to the health adviser agent 110, enabling continuous updating of the user body model 114 and refinement of the training plan 110a based on real-world performance and feedback. This closed-loop architecture supports adaptive, user-specific optimization of physical support and training, ensuring that the system 100 can respond dynamically to changes in the user's health, fitness, and activity context.

The system 100 distinguishes between a long-term training planner 116, located within the health adviser agent 110, which generates broad, scheduled training plans (e.g., weekly routines), and a real-time stimulation planner 124, located within the activity agent 120, which performs ad hoc adjustments to support levels during ongoing activities.

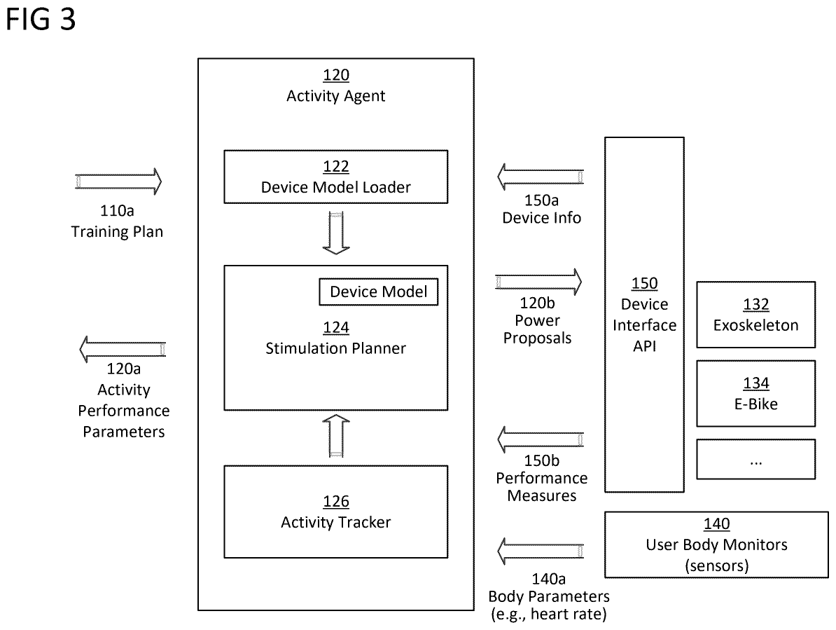

FIG. 3 illustrates a block diagram of the activity agent 120 system architecture, detailing the flow of information and control between its internal components, external devices, and monitoring systems. The activity agent 120 is responsible for both enabling the desired stimulation of the user's body and tracking the user's activity and stimulation in real-time. The activity agent 120 can connect to various body-support devices 130, such as an exoskeleton 132 or an electrically-assisted bike 134, to support human movement.

To create the correct stimulation for the user, the activity agent 120 includes a device model loader 122, which retrieves a device model that represents operational characteristics of the body-support device 130 or other body support device information 150a from a database based on an identification signal received from the body-support device 130 or directly from the connected body-support device 130. The device model loader 122 loads this information into a device model, which characterizes the operational parameters and capabilities of the specific device in use.

The device model is then provided to a stimulation planner 124 within the activity agent 120. The stimulation planner 124 executes the training plan 110a received from the health adviser agent 110, for example, by defining the power a given muscular group of the user should currently perform or, conversely, the power the device should provide to support the user's body. The stimulation planner 124 also uses real-time activity performance parameters from the activity tracker 126 to determine the target level and distribution of support to be provided by the body-support device.

The activity tracker 126 continuously monitors performance measures 150b from the body-support device 130 and physiological parameters 140a from human body monitoring 140, such as heart rate, blood pressure, and other relevant metrics. The activity tracker 126 can also process fine-grained and dynamic data, such as power data from both pedals of an e-bike, to detect sudden weakness in one limb or other asymmetries that may require additional support. The activity tracker 126 calculates the force and work that the user's body or muscles have performed or are performing. This information is tracked and reported back to the stimulation planner 124.

Based on the aggregated data, the stimulation planner 124 generates power proposals 120b, which are transmitted via the application programming interface (API) 150 to the body-support devices 130 to control the level of assistance provided. These power proposals 120b serve as control signals that instruct the body-support devices 130 to adjust their operation and provide the appropriate level of support to the user.

The activity agent 120 also receives continuous feedback from the devices 130 and monitoring systems 140, enabling dynamic, closed-loop adjustments of support in real-time. For example, if the activity has just started, the support of the body-support device 130 might be lowered; if the body is already fatigued or it is foreseeable that the ideal dose of stress is about to be reached, the assistance will be increased.

The stimulation planner 124 generates power proposals 120b that may specify support levels for individual actuators, such as motors in an electrically-assisted bicycle or joints in an exoskeleton. For example, the system can allocate a specific amount of support power to the right pedal motor of an e-bike if a temporary weakness is detected in the user's right leg, or adjust the torque provided to a particular joint in an exoskeleton to compensate for localized fatigue or injury. This fine-grained, targeted allocation of support enables the system 100 to dynamically tailor assistance to the user's real-time physiological needs and detected asymmetries.

The stimulation planner 124 generates a control signal that encodes the desired support parameters, such as power, force, or torque, to be applied by the body-support device 130. This control signal may specify support levels for individual actuators or joints, and is transmitted to the body-support device 130 via the API 150. Upon receiving the control signal, the body-support device 130 adjusts its operation accordingly to provide the target level of physical support to the user.

The system 100 may comprise a user interface that enables the user to manually override, adjust, or select the target level of physical support provided by the body-support device 130. The user interface may be implemented on a smartphone, wearable device, or integrated into the body-support device 130 itself, allowing the user to adjust the support level, select from predefined support modes, or temporarily disable automated adjustments as desired.

The system 100 may be further configured to detect an emergency condition or anomalous physiological state in the user, such as abnormally high or low heart rate, loss of consciousness, or other critical physiological events, based on real-time data from the user body monitors 140. Upon detection of such a condition, the activity agent 120 may modify or disable the target level of physical support provided by the body-support device 130, for example, by increasing support to prevent user collapse, reducing support to avoid further stress, or stopping device operation to ensure user safety.

Additionally, the activity agent 120 communicates activity performance parameters 120a and performs activities in response to the health adviser agent 110, supporting the continuous updating of the user body model 114 and refinement of the training plan 110a. This architecture enables the system 100 to provide adaptive, user-specific physical support that responds dynamically to the user's physiological state and activity context.

A benefit of the disclosed system 100 is the tight integration of the stimulation planner 124 with real-time feedback from the activity tracker 126. This architecture enables the system 100 to respond dynamically to the user's actual fitness and performance level, rather than relying solely on static plans or assumptions. For instance, if the user's fitness on a particular day is lower than expected, due to factors such as poor sleep or elevated ambient temperature, the system 100 can immediately adjust the training plan 110a. If the training plan 110a specifies a target heart rate of 120 beats per minute, the system 100 can use the user body model 114 to forecast the required assistance power. Should the user's heart rate rise unexpectedly, indicating increased exertion, the planner can increase the support power in real time to help maintain the desired heart rate. This adaptive approach ensures desired training intensity, user safety, and increases the health benefits of body-support devices.

In certain aspects, the system 100 may utilize the user body model 114 to predict the user's future physiological state and preemptively adjust the target level of physical support to prevent physiological overload, fatigue, injury, or to enhance comfort or performance.

As a non-limiting example, consider a scenario in which a user's fitness tracker indicates an overall good physical condition. However, due to a recent football match, the user experiences pain in the right knee and feels discomfort when performing pedal strokes with the right leg on an electrically-assisted bike. The system described herein can detect this asymmetry in physiological performance between the user's body parts using power sensors in the pedals, which identify reduced force or irregular movement on the right side. In response, the system dynamically increases support for the right leg while maintaining the appropriate level of effort for the left leg, thereby compensating for the temporary weakness. This targeted, real-time adjustment ensures that the user continues to receive desired support and training benefits, even in the presence of transient injuries or fatigue, capabilities not provided by existing systems.

In some aspects, the user body model 114, training planner 114, or stimulation planner 124 may be implemented as an artificial intelligence (AI) model or agent. The AI model may take the form of a neural network, a decision tree, a support vector machine, a knowledge-based system, or a large language model. The model may be trained using supervised, unsupervised, or reinforcement learning techniques, and may be updated continuously or periodically based on real-time physiological data and user performance history. The AI model receives as input user-specific physiological parameters, activity data, and defined goals 112, and outputs a training plan 110a or real-time support adjustment parameters tailored to the user's current or predicted state.

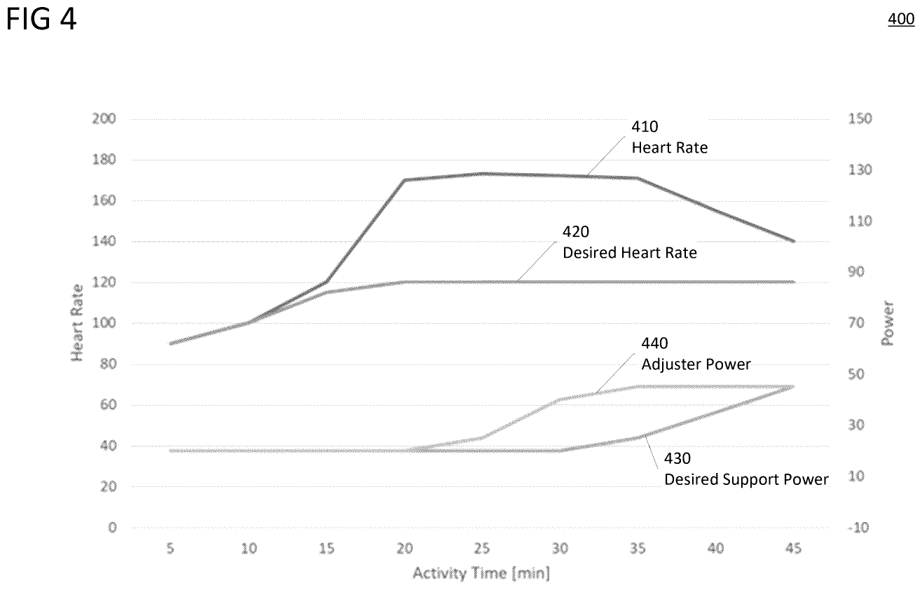

FIG. 4 presents a graph 400 illustrating the relationship between heart rate 410, desired heart rate 420, desired support power 430, and adjuster power 440 during an activity session. The horizontal axis represents activity time in minutes, while the left vertical axis indicates heart rate (in beats per minute) and the right vertical axis indicates power (in watts).

Line 410 depicts the user's actual heart rate measured during the activity. Line 420 represents the desired heart rate, as specified by the training plan 110a or system target. Line 430 shows the desired support power, which is the level of assistance the system 100 aims to provide through the body-support device 130 to help the user achieve the desired physiological state. Line 440 indicates the adjuster power, reflecting real-time adjustments made by the system 100 to the support power in response to deviations between the actual and desired heart rate.

As shown in graph 400, the system 100 continuously monitors the user's physiological state and dynamically adjusts the support power to maintain the heart rate within the desired range. When the user's heart rate exceeds the target, the system 100 increases the support power (as indicated by the adjuster power line 440) to reduce exertion. Conversely, if the heart rate falls below the desired level, the system 100 may decrease support to encourage greater physical effort. This closed-loop feedback mechanism enables the system 100 to optimize training intensity and user safety in real time.

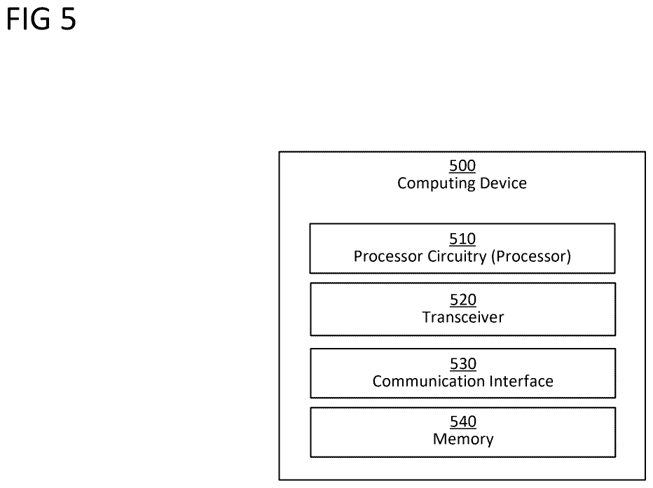

FIG. 5 illustrates a computing device 500 in accordance with aspects of the disclosure.

The computing device 500 may be identified with a central controller and implemented as any suitable network infrastructure component, such as a cloud or cloud edge network server, controller, or computing device. The computing device 500 may serve the health advisor agent 110 and/or the activity agent 120, in accordance with the various techniques discussed herein. To do so, the computing device 500 may include processor circuitry 510, a transceiver 520, a communication interface 530, and a memory 540. The components shown in FIG. 5 are provided for ease of explanation, and the computing device 500 may implement additional, fewer, or alternative components than those shown in FIG. 5.

The processor circuitry 510 may be operable as any suitable number and/or type of computer processor that may function to control the computing device 500. The processor circuitry 510 may be identified with at least one processor (or suitable portions thereof) implemented by the computing device 500. The processor circuitry 510 may be identified with at least one processor, such as a host processor, a digital signal processor, one or more microprocessors, graphics processors, baseband processors, microcontrollers, an application-specific integrated circuit (ASIC), a portion (or the entirety of) a field-programmable gate array (FPGA), etc.

In any case, the processor circuitry 510 may be operable to execute instructions to perform arithmetic, logic, and/or input/output (I/O) operations and/or to control the operation of one or more components of the computing device 500 to perform various functions as described herein. The processor circuitry 510 may include one or more microprocessor cores, memory registers, buffers, and clocks, among other components. It may generate electronic control signals associated with the components of the computing device 500 to control and/or modify the operation of those components. The processor circuitry 510 may communicate with and/or control functions associated with the transceiver 520, the communication interface 530, and/or the memory 540. The processor circuitry 510 may also perform various operations to control communications, scheduling, and/or the operation of other network infrastructure components communicatively coupled to the computing device 500.

The transceiver 520 may be implemented as any suitable number and/or type of components capable of transmitting and/or receiving data packets and/or wireless signals in accordance with any suitable number and/or type of communication protocols. The transceiver 520 may include any suitable type of components to facilitate this functionality, including components associated with known transceiver, transmitter, and/or receiver operations, configurations, and implementations. Although depicted as a transceiver in FIG. 5, the transceiver 520 may comprise any suitable number of transmitters, receivers, or combinations thereof, which can be integrated into a single transceiver or as multiple transceivers or transceiver modules. The transceiver 520 may include components typically identified with a radio frequency (RF) front end and include, for example, antennas, ports, power amplifiers (PAs), RF filters, mixers, local oscillators (LOs), low noise amplifiers (LNAs), up-converters, down-converters, channel tuners, etc.

The communication interface 530 may be implemented as any suitable number and/or type of components operable to facilitate the transceiver 520 to receive and/or transmit data and/or signals in accordance with one or more communication protocols, as discussed herein. The communication interface 530 may be implemented as any suitable number and/or type of components operable to interface with the transceiver 520, such as analog-to-digital converters (ADCs), digital-to-analog converters, intermediate frequency (IF) amplifiers and/or filters, modulators, demodulators, baseband processors, and the like. The communication interface 530 may thus operate in conjunction with the transceiver 520 and form part of an overall communication circuitry implemented by the computing device 500, which may be implemented via the computing device 500 to transmit commands and/or control signals to perform any of the functions described herein.

The memory 540 is operable to store data and/or instructions such that when the instructions are executed by the processor circuitry 510, they cause the computing device 500 to perform various functions as described herein. The memory 540 may be implemented as any known volatile and/or non-volatile memory, including, for example, read-only memory (ROM), random access memory (RAM), flash memory, a magnetic storage medium, an optical disk, erasable programmable read-only memory (EPROM), programmable read-only memory (PROM), etc. The memory 540 may be non-removable, removable, or a combination of the two. The memory 540 may be implemented as a non-transitory computer-readable medium that stores one or more executable instructions, such as logic, algorithms, or code.

As further discussed below, the instructions, logic, code, etc., stored in the memory 540 are represented by the various modules/engines as shown in FIG. 5. Alternatively, when implemented via hardware, the modules/engines shown in FIG. 5 associated with the memory 540 may include instructions and/or code to facilitate control and/or monitoring of the operation of such hardware components. In other words, the modules/engines shown in FIG. 5 are provided to facilitate an explanation of the functional association between hardware and software components. Thus, the processor circuitry 510 may execute the instructions stored in these respective modules/engines in conjunction with one or more hardware components to perform the various functions discussed herein.

Various aspects described herein may utilize one or more machine learning models for the health advisor agent 110 and/or the activity agent 120. The term “model,” as used herein, may be understood to mean any algorithm that provides output data from input data (e.g., any type of algorithm that generates or calculates output data from input data). A machine learning model can be executed by a computing system to improve the performance of a particular task progressively. In some aspects, the parameters of a machine learning model can be adjusted during the training phase based on the training data. A trained machine learning model may be used during an inference phase to make predictions or decisions based on input data. In some aspects, the trained machine learning model may be used to generate additional training data. An additional machine learning model may be tuned during a second training phase based on the generated additional training data. A trained additional machine learning model may be used during an inference phase to make predictions or decisions based on input data.

The machine learning models described herein may take any suitable form or utilize any suitable technique (e.g., for training purposes). For example, each of the machine learning models may utilize supervised learning, semi-supervised learning, unsupervised learning, or reinforcement learning techniques.

In supervised learning, the model is built using a training set of data that includes both the inputs and the corresponding desired outputs (illustratively, each input is associated with a desired or expected output for that input). Each training instance may include one or more inputs and a desired output. Training may involve iterating through training instances and using an objective function to teach the model to predict the output for new inputs (illustratively, for inputs not included in the training set). In semi-supervised learning, a portion of the inputs in the training set may lack corresponding desired outputs (e.g., one or more inputs may not be associated with any desired or expected output).

In unsupervised learning, the model is built from a training set of data that includes only inputs, without any desired outputs. The unsupervised model can be used to identify structure in the data (e.g., grouping or clustering of data points), for example, by discovering patterns within the data. Techniques that may be implemented in an unsupervised learning model include self-organizing maps, nearest-neighbor mapping, k-means clustering, and singular value decomposition.

Reinforcement learning models may include positive or negative feedback to improve accuracy. A reinforcement learning model may attempt to increase one or more goals/rewards. Techniques that can be implemented in a reinforcement learning model include, for example, Q-learning, temporal difference (TD) learning, and deep adversarial networks.

Various aspects described herein may utilize one or more classification models. In a classification model, outputs may be restricted to a limited set of values (e.g., one or more classes). The classification model may output a class for an input set of one or more input values. An input set may include sensor data, such as image data, radar data, LIDAR (light detection and ranging) data, and the like. A classification model as described herein may, for example, classify certain driving conditions and/or environmental conditions, such as weather conditions, road conditions, and the like. References herein to classification models may encompass a model that implements, for example, one or more of the following techniques: linear classifiers (e.g., logistic regression or naive Bayes classifier), support vector machines, decision trees, boosted trees, random forests, neural networks, or nearest neighbors.

Various aspects described herein may utilize one or more regression models. A regression model may output a numerical value from a continuous range based on an input set of one or more values (e.g., starting from or using an input set of one or more values). References herein to regression models may contemplate a model that implements, for example, one or more of the following techniques (or other suitable techniques): linear regression, decision trees, random forests, or neural networks.

A machine learning model described herein may be or include a neural network. The neural network may be any type of neural network, such as a convolutional neural network, an autoencoder network, a variational autoencoder network, a sparse autoencoder network, a recurrent neural network, a deconvolutional network, a generative adversarial network, a forward-thinking neural network, a sum-product neural network, and the like. The neural network can have any number of layers. The training of the neural network (e.g., adapting the network's layers) may utilize or be based on any training principle, such as backpropagation (e.g., employing the backpropagation algorithm).

The techniques described in this disclosure may also be illustrated in the following examples.
Example 1. A system, comprising: at least one processor configured to: receive real-time data indicative of a physiological state of a user during operation of a body-support device; determine, based at least in part on the real-time data and a training plan generated from a user body model representing a current physiological state of the user, a target level of physical support for the user; generate a control signal corresponding to the target level of physical support; and dynamically adjust the control signal in response to detected changes in the user's physiological state, as indicated by the real-time data, during operation of the body-support device; and an interface configured to transmit the control signal to the body-support device to dynamically provide the target level of physical support to the user.
Example 2. The system of example 1, wherein the at least one processor is further configured to generate the training plan based at least in part on the user body model that is user-specific, population-based, or adaptively updated, and at least one defined goal.
Example 3. The system of any one or more examples 1-2, wherein the at least one processor is further configured to: detect an asymmetry in physiological performance between body parts of the user based on the real-time data; and adjust the target level of physical support asymmetrically to compensate for the asymmetry.
Example 4. The system of any one or more examples 1-3, wherein the at least one processor is further configured to access a device model that represents operational characteristics of the body-support device and to use the device model to determine how to apply the control signal to achieve the target level of physical support.
Example 5. The system of any one or more examples 1-4, wherein the body-support device comprises an exoskeleton or an electrically-assisted bicycle.
Example 6. The system of any one or more examples 1-5, wherein the at least one processor is located at a cloud edge.
Example 7. The system of any one or more examples 1-6, wherein the at least one processor is further configured to execute a health adviser agent configured to maintain the user body model and generate the training plan based at least in part on at least one defined goal.
Example 8. The system of any one or more examples 1-7, wherein the at least one processor is further configured to update the user body model based on real-time data and a performance history of the user during operation of the body-support device.
Example 9. The system of any one or more examples 1-8, wherein the at least one processor is further configured to retrieve a device model from a database based on an identification signal received from the body-support device.
Example 10. The system of any one or more examples 1-9, wherein the at least one processor is further configured to predict a future physiological state of the user and adjust the target level of physical support preemptively to avoid physiological overload, fatigue, injury, or to improve comfort or performance.
Example 11. The system of any one or more examples 1-10, wherein the real-time data comprises data sensed by a heart rate sensor, electrocardiogram (ECG) sensor, blood pressure sensor, blood oxygenation (SpO2) sensor, temperature sensor, respiratory rate sensor, electromyography (EMG) sensor, accelerometers, gyroscope, force sensor, or power sensor.
Example 12. The system of any one or more examples 1-11, further comprising a user interface configured to enable the user to manually override, adjust, or select the target level of physical support.
Example 13. The system of any one or more examples 1-12, wherein the at least one processor is further configured to detect or respond to an emergency condition or anomalous physiological state by modifying or disabling the target level of physical support provided by the body-support device.
Example 14. A non-transitory computer-readable medium storing instructions that, when executed by at least one processor, cause the processor to perform operations comprising: receiving real-time data indicative of a physiological state of a user during operation of a body-support device; determining, based at least in part on the real-time data and a training plan generated from a user body model representing a current physiological state of the user, a target level of physical support for the user; generating a control signal corresponding to the target level of physical support; dynamically adjusting the control signal in response to detected changes in the user's physiological state, as indicated by the real-time data, during operation of the body-support device; and transmitting the control signal to the body-support device to dynamically provide the target level of physical support to the user.
Example 15. The non-transitory computer-readable medium of example 14, wherein the instructions further cause the processor to generate the training plan based at least in part on the user body model that is user-specific, population-based, or adaptively updated, and at least one defined goal.
Example 16. The non-transitory computer-readable medium of any one or more examples 14-15, wherein the instructions further cause the processor to: detect an asymmetry in physiological performance between body parts of the user based on the real-time data; and adjust the target level of physical support asymmetrically to compensate for the asymmetry.
Example 17. The non-transitory computer-readable medium of any one or more examples 14-16, wherein the instructions further cause the processor to access a device model that represents operational characteristics of the body-support device and use the device model to determine how to apply the control signal to achieve the target level of physical support.
Example 18. The non-transitory computer-readable medium of any one or more examples 14-17, wherein the instructions further cause the processor to predict a future physiological state of the user and adjust the target level of physical support preemptively to avoid physiological overload, fatigue, injury, or to improve comfort or performance.
Example 19. The non-transitory computer-readable medium of any one or more examples 14-18, wherein the instructions further cause the processor to update the user body model based on real-time data and a performance history of the user during operation of the body-support device.
Example 20. The non-transitory computer-readable medium of any one or more examples 14-19, wherein the real-time data comprises data sensed by a heart rate sensor, electrocardiogram (ECG) sensor, blood pressure sensor, blood oxygenation (SpO2) sensor, temperature sensor, respiratory rate sensor, electromyography (EMG) sensor, accelerometers, gyroscope, force sensor, or power sensor.

Although specific aspects have been illustrated and described herein, it will be appreciated by those of ordinary skill in the art that a variety of alternate and/or equivalent implementations may be substituted for the specific aspects shown and described without departing from the scope of the present application. This application is intended to cover any adaptations or variations of the specific aspects discussed herein.

1. A system, comprising:
at least one processor configured to:
receive real-time data indicative of a physiological state of a user during operation of a body-support device;
determine, based at least in part on the real-time data and a training plan generated from a user body model representing a current physiological state of the user, a target level of physical support for the user;
generate a control signal corresponding to the target level of physical support; and
dynamically adjust the control signal in response to detected changes in the user's physiological state, as indicated by the real-time data, during operation of the body-support device; and
an interface configured to transmit the control signal to the body-support device to dynamically provide the target level of physical support to the user.

2. The system of claim 1, wherein the at least one processor is further configured to generate the training plan based at least in part on the user body model that is user-specific, population-based, or adaptively updated, and at least one defined goal.

3. The system of claim 1, wherein the at least one processor is further configured to:
detect an asymmetry in physiological performance between body parts of the user based on the real-time data; and
adjust the target level of physical support asymmetrically to compensate for the asymmetry.

4. The system of claim 1, wherein the at least one processor is further configured to access a device model that represents operational characteristics of the body-support device and to use the device model to determine how to apply the control signal to achieve the target level of physical support.

5. The system of claim 1, wherein the body-support device comprises an exoskeleton or an electrically-assisted bicycle.

6. The system of claim 1, wherein the at least one processor is located at a cloud edge.

7. The system of claim 1, wherein the at least one processor is further configured to execute a health adviser agent configured to maintain the user body model and generate the training plan based at least in part on at least one defined goal.

8. The system of claim 1, wherein the at least one processor is further configured to update the user body model based on real-time data and a performance history of the user during operation of the body-support device.

9. The system of claim 1, wherein the at least one processor is further configured to retrieve a device model from a database based on an identification signal received from the body-support device.

10. The system of claim 1, wherein the at least one processor is further configured to predict a future physiological state of the user and adjust the target level of physical support preemptively to avoid physiological overload, fatigue, injury, or to improve comfort or performance.

11. The system of claim 1, wherein the real-time data comprises data sensed by a heart rate sensor, electrocardiogram (ECG) sensor, blood pressure sensor, blood oxygenation (SpO2) sensor, temperature sensor, respiratory rate sensor, electromyography (EMG) sensor, accelerometers, gyroscope, force sensor, or power sensor.

12. The system of claim 1, further comprising a user interface configured to enable the user to manually override, adjust, or select the target level of physical support.

13. The system of claim 1, wherein the at least one processor is further configured to detect or respond to an emergency condition or anomalous physiological state by modifying or disabling the target level of physical support provided by the body-support device.

14. A non-transitory computer-readable medium storing instructions that, when executed by at least one processor, cause the processor to perform operations comprising:
receiving real-time data indicative of a physiological state of a user during operation of a body-support device;
determining, based at least in part on the real-time data and a training plan generated from a user body model representing a current physiological state of the user, a target level of physical support for the user;
generating a control signal corresponding to the target level of physical support;
dynamically adjusting the control signal in response to detected changes in the user's physiological state, as indicated by the real-time data, during operation of the body-support device; and
transmitting the control signal to the body-support device to dynamically provide the target level of physical support to the user.

15. The non-transitory computer-readable medium of claim 14, wherein the instructions further cause the processor to generate the training plan based at least in part on the user body model that is user-specific, population-based, or adaptively updated, and at least one defined goal.

16. The non-transitory computer-readable medium of claim 14, wherein the instructions further cause the processor to:
detect an asymmetry in physiological performance between body parts of the user based on the real-time data; and
adjust the target level of physical support asymmetrically to compensate for the asymmetry.

17. The non-transitory computer-readable medium of claim 14, wherein the instructions further cause the processor to access a device model that represents operational characteristics of the body-support device and use the device model to determine how to apply the control signal to achieve the target level of physical support.

18. The non-transitory computer-readable medium of claim 14, wherein the instructions further cause the processor to predict a future physiological state of the user and adjust the target level of physical support preemptively to avoid physiological overload, fatigue, injury, or to improve comfort or performance.

19. The non-transitory computer-readable medium of claim 14, wherein the instructions further cause the processor to update the user body model based on real-time data and a performance history of the user during operation of the body-support device.

20. The non-transitory computer-readable medium of claim 14, wherein the real-time data comprises data sensed by a heart rate sensor, electrocardiogram (ECG) sensor, blood pressure sensor, blood oxygenation (SpO2) sensor, temperature sensor, respiratory rate sensor, electromyography (EMG) sensor, accelerometers, gyroscope, force sensor, or power sensor.
